(19) United States
(12) Patent Application Publication
(10) Pub. No.: US 2005/0020589 A1
(43) Pub. Date: Jan. 27, 2005

(54) Sustained-release tablet composition comprising a dopamine receptor agonist

(75) Inventors: Loksidh D. Ganorkar, (US); Joseph P. Reo, (US); Alice C. Martino, Kalamazoo, MI (US); Gregory E. Amidon, (US); Connie J. Skoug, (US)

Correspondence Address: PFIZER INC, 150 EAST 42ND STREET, 5TH FLOOR - STOP 49, NEW YORK, NY, 10017-5612, US

(73) Assignee: Pfizer Inc.

(21) Appl. No.: 10/871,646

(22) Filed: Jun. 18, 2004

Related U.S. Application Data
(60) Provisional application No. 60/479,327, filed on Jun. 18, 2003.

(51) Int. Cl. A61K 31/538; A61K 31/5377; A61K 31/498; A61K 31/496

(52) U.S. Cl. 514/229.8; 514/250; 514/233.2; 514/253.03; 514/296; 514/60

(57) ABSTRACT

A sustained-release pharmaceutical composition in a form of an orally deliverable tablet comprises as active pharmaceutical agent a compound of formula 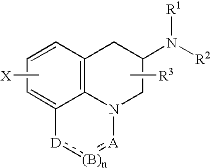
or a pharmaceutically acceptable salt thereof, wherein R¹, R² and R³ are the same or different and are H, C₁₋₆ alkyl (optionally phenyl substituted), C₃₋₅ alkenyl or alkynyl or C₃₋₁₀ cycloalkyl, or where R³ is as above and R¹ and R² are cyclized with the attached N atom to form pyrrolidinyl, piperidinyl, morpholinyl, 4-methylpiperazinyl or imidazolyl groups; X is H, F, Cl, Br, I, OH, C₁₋₆ alkyl or alkoxy, CN, carboxamide, carboxyl or (C₁₋₆ alkyl)carbonyl; A is CH, CH₂, CHF, CHCl, CHBr, CHI, CHCH₃, C═O, C═S, CSCH₃, C═NH, CNH₂, CNHCH₃, CNHCOOCH₃, CNHCN, SO₂ or N; B is CH, CH₂, CHF, CHCl, CHBr, CHI, C═O, N, NH or NCH₃, and n is 0 or 1; and D is CH, CH₂, CHF, CHCl, CHBr, CHI, C═O, O, N, NH or NCH₃. The agent is. dispersed in a matrix comprising a hydrophilic polymer and a starch having a tensile strength of at least about 0.15 kN cm⁻² at a solid fraction representative of the tablet. The composition exhibits sustained-release properties effective for treatment of Parkinson's disease. The tablet is optionally coated. Tablets of the invention have improved resistance to attrition or erosion during manufacture, packaging and handling.

### FIELD OF THE INVENTION

The present invention relates to tablet formulations, and more particularly to a sustained-release tablet composition for oral delivery of a water-soluble dopamine receptor agonist.

### BACKGROUND OF THE INVENTION

Many active pharmaceutical agents, including drugs and prodrugs, have been formulated as orally deliverable dosage forms providing sustained release (otherwise known as slow release or extended release) of such agents over a period of time effective to permit once daily administration. A well-known system for formulating such dosage forms involves a matrix comprising a hydrophilic polymer wherein the agent is dispersed; the agent is released over a period of time in the gastrointestinal tract upon dissolution or erosion ofthe matrix. Sustained-release dosage forms comprising such a matrix system are conveniently prepared as compressed tablets, described herein as “matrix tablets”.

Drugs and prodrugs having relatively high solubility in water, for example a solubility of about 10 mg/ml or greater, present challenges to the formulator wishing to provide a sustained-release dosage form, and the higher the solubility the greater are the challenges. These challenges are well illustrated in the case of sumanirole maleate.

Sumanirole is a highly selective dopamine D₂ receptor agonist useful in treatment of a variety of conditions and disorders of the central nervous system (CNS) including Parkinson's disease, restless legs syndrome and sexual dysfunction. The maleate salt of sumanirole has been selected based on its physical and chemical properties. This salt is highly soluble.

U.S. Pat. No. 6,197,339 discloses a sustairied-release tablet comprising (R)-5,6-dihydro-5-(methylarnino)-4H-imidazo[4,5-ij]-quinolin-2(1H)-one (Z)-2-butenedioate (1:1) (sumanirole maleate) in a matrix comprising hydroxypropylmethylcellulose (HPMC) and starch. The tablet is disclosed to be useful in treatment of Parkinson's disease. Starches disclosed to be suitable therein include pregelatinized starch.

European Patent Application No. EP 0 933 079 discloses a starch said to be suitable for preparing tablets having high hardness yet being capable of rapid disintegration in an aqueous medium. Tensile strength of the finished tablets is calculated from the hardness.

Patents and publications cited above are incorporated herein by reference.

Tablets prepared as described in above-cited U.S. Pat. No. 6,197,339 exhibit good therapeutic effectiveness but can be susceptible to attrition and/or erosion during manufacture, packaging and handling.

It is an object of the present invention to provide a sustained-release tablet composition of a water-soluble dop amine receptor agonist, the tablet having sufficient hardness to withstand a high-speed tableting and/or coating operation, in particular to resist erosion during such an operation.

### SUMMARY OF THE INVENTION

There is now provided a sustained-release pharmaceutical composition in a form of an orally deliverable tablet comprising as active pharmaceutical agent a compound of formula (I) 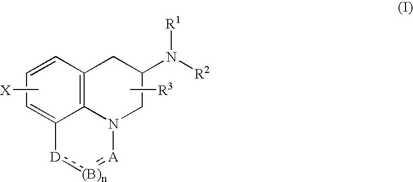
or a pharmaceutically acceptable salt thereof, wherein

R¹, R² and R³ are the same or different and are H, C₁₋₆ alkyl (optionally phenyl substituted), C₃₋₅ alkenyl or alkynyl or C₃₋₁₀ cycloalkyl, or where R³ is as above and R¹ and R² are cyclized with the attached N atom to form pyrrolidinyl, piperidinyl, morpholinyl, 4-methylpiperazinyl or imidazolyl groups;

X is H, F, Cl, Br, I, OH, C₁₋₆ alkyl or alkoxy, CN, carboxamide, carboxyl or (C₁₋₆ alkyl)carbonyl;

A is CH, CH₂, CHF, CHCl, CHBr, CHI, CHCH₃, C═O, C═S, CSCH₃, C═NH, CNH₂, CNHCH₃, CNHCOOCH₃, CNHCN, SO₂ or N;

B is CH, CH₂, CHF, CHCl, CHBr, CHI, C═O, N, NH or NCH₃, and n is 0 or 1; and

D is CH, CH₂, CHF, CHCl, CHBr, CHI, C═O, O, N, NH or NCH₃.

It is preferred that the compound of formula (I) or salt thereof has water solubility of at least about 10 mg/ml, more preferably at least about 50 mg/ml and most preferably at least about 100 mg/ml.

The active pharmaceutical agent is dispersed in a matrix comprising a hydrophilic polymer and a starch having a tensile strength of at least about 0.15 kN cm⁻² at a solid fraction representative of the tablet.

There is further provided a process for preparing a sustained-release pharmaceutical composition in a form of an orally deliverable tablet comprising as active pharmaceutical agent a compound of formula (I) or a salt thereof, the process comprising selecting by a suitable test a starch having a tensile strength of at least about 0.15 kN cm⁻² at a solid fraction representative of the tablet; admixing with the selected starch a hydrophilic polymer and the agent to provide a mixture wherein the agent is dispersed in a matrix comprising the polymer and the starch; and compressing the mixture to form a tablet.

A particularly convenient test method, which is itself a further embodiment of the invention, comprises preparing compacts of a starch sample on an automated tablet press at a range of compression forces, measuring hardness of the compacts, determining solid fraction of the compacts, calculating tensile strength of the compacts from hardness and dimensions of the compacts, determining relationship of tensile strength to solid fraction of the compacts, and from that relationship estimating tensile strength at a solid fraction representative of a desired tablet.

There is still further provided a method of treatment of a subject having a condition or disorder for which a dopamine agonist is indicated, the method comprising orally administering to the subject a sustained-release pharmaceutical composition in a form of a tablet comprising as active pharmaceutical agent a compound of formula (I) or salt thereof, dispersed in a matrix comprising a hydrophilic polymer and a starch having a tensile strength of at least about 0.15 kN cm⁻² at a solid fraction representative of the tablet.

An “active pharmaceutical agent” herein can be a drug or a prodrug or a salt thereof, including diagnostic agents. Unless otherwise specified, “solubility” herein means solubility in water at 20-25° C. at any physiologically acceptable pH, for example at any pH in the range of about 4 to about 8. In the case of an agent that is a salt, reference herein to solubility in water pertains to the salt, not to the free acid or base form of the agent.

The term “orally deliverable” herein means suitable for oral, including peroral and intra-oral (e.g., sublingual or buccal) administration, but tablets of the present invention are adapted primarily for peroral administration, i.e., for swallowing, typically whole and with the aid of water or other drinkable fluid.

A “compact” herein is a compressed tablet, prepared for example on a tablet press, consisting only of a sample of starch for which it is desired to measure tensile strength. “Solid fraction” is the ratio of absolute to apparent density of a compact. A “solid fraction representative of the tablet” is a solid fraction selected to be similar to the solid fraction of tablets prepared according to the invention. Typically a solid fraction of about 0.75 to about 0.85, illustratively 0.8, will be selected.

A “subject” herein is an animal of any species, preferably mammalian, most preferably human. Conditions and disorders in a subject for which a particular agent is said herein to be “indicated” are not restricted to conditions and disorders for which the agent has been expressly approved by a regulatory authority, but also include other conditions and disorders known or believed by a physician to be amenable to treatment with the agent. “Treatment” herein embraces prophylactic treatment unless the context requires otherwise.

### BRIEF DESCRIPTION OF THE DRAWINGS

FIG. 1 is a graph showing relationship of tensile strength of pregelatinized starch lots, as determined by a test method of the invention using a 4 second dwell time (Example 1 herein) to triaxial tensile strength.

FIG. 2 is a graph showing relationship of tensile strength of pregelatinized starch lots, as determined by a test method of the invention using a 90 second dwell time (Example 1 herein) to triaxial tensile strength.

FIG. 3 is a graph showing correlation oftensile strength of pregelatinized starch lots with maximum hardness oftablets containing these lots.

### DETAILED DESCRIPTION OF THE INVENTION

In one embodiment, the invention provides a pharmaceutical composition in a form of an orally deliverable tablet comprising as active pharmaceutical agent a compound of formula (I) or a salt thereof.

Pharmaceutically acceptable salts of a compound of formula (I) include without restriction salts of the following acids: hydrochloric, hydrobromic, hydroiodic, sulfuric, methanesulfonic, ethanesulfonic, benzenesulfonic, p-toluenesulfonic, phosphoric, nitric, lactic, malic, benzoic, citric, tartaric, fumaric and maleic acids, and mono- and dicarboxylic acids of formulas CH₃—(CH₂)ₙ—COOH and HOOC—(CH₂)ₙ—COOH where n is 0 to 4, for example acetic, propionic, malonic and succinic acids.

Particularly preferred salts are the hydrochloride salt and the maleate, i.e., (Z)-2-butenedioate, salt.

Compounds of formula (I) and their salts can be prepared by processes known per se, including processes described in patent literature cited herein. However, the present invention is not restricted by the process used to prepare the therapeutic agent.

Preferred compounds of formula (I) include those disclosed generically or specifically in U.S. Pat. No. 5,273,975, which is incorporated herein by reference. Especially preferred compounds are salts of sumanirole, in the form of its R-enantiomer, (R)-5,6-dihydro-5-(methylamino)-4H-imidazo[4,5-ij]-quinolin-2(1H)-one (II), and its thione counterpart (R)-5,6-dihydro-5-(methylamino)-4H-imidazo[4,5-ij]-quinoline-2(1H)-thione (III).

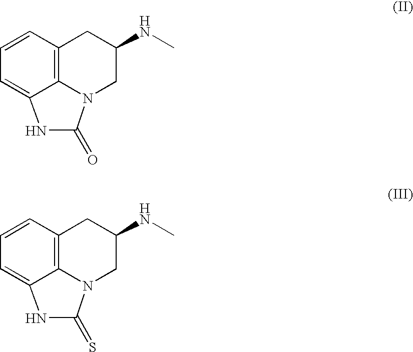

In the case of either compound (II) or (III), suitable salts include hydrochloride, hydrobromide, hydroiodide, sulfate, phosphate, acetate, propionate, lactate, maleate, malate, succinate, tartrate, cyclohexanesulfamate, mesylate (methanesulfonate), esylate (ethanesulfonate), besylate (benzenesulfonate) and tosylate (p-toluenesulfonate) salts. The maleate salt is preferred. Use of this salt in treatment of restless legs syndrome is specifically disclosed in International Patent Publication No. WO 02/36123.

The amount of the active pharmaceutical agent present in a composition of the invention depends on the potency of the agent, but is preferably sufficient to provide a daily dose in one to a small plurality, for example one to about 4, of tablets to be administered no more than twice daily. Preferably a single tablet provides a sufficient amount of the agent for each administration. In most cases the amount of the agent per tablet is about 0.1 to about 200 mg, preferably about 0.2 to about 100 mg. Expressed as percentage by weight of the composition, the amount of the agent is typically about 0.01% to about 25%, preferably about 0.05% to about 20%. In the case of an agent that is a salt, amounts of agent herein are expressed as free acid or free base equivalent amounts, unless otherwise specified.

Illustratively in the case of sumanirole, an amount of about 0.5 to about 25 mg per tablet, or about 0.1% to about 15% by weight of the composition, will generally be suitable. Specific dosage amounts per tablet contemplated herein include 0.5, 1, 2, 4, 8, 12 and 24 mg sumanirole in the form of sumanirole maleate.

A composition of the present invention comprises an active pharmaceutical agent as defined above, dispersed in a matrix comprising a hydrophilic polymer and a starch having a tensile strength of at least about 0.15 kN cm⁻² at a solid fraction representative of the tablet, for example about 0.75 to about 0.85, illustratively 0.8.

Hydrophilic polymers useful herein are pharmaceutically acceptable polymeric materials having a sufficient number and distribution of hydrophilic substituents such as hydroxy and carboxy groups to impart hydrophilic properties to the polymer as a whole. Suitable hydrophilic polymers include, without limitation, methylcellulose, HPMC (hypromellose), carmellose (carboxymethylcellulose) sodium and carbomer (polyacrylic acid). More than one such polymer can optionally be used.

HPMC is a preferred hydrophilic polymer. Various types and grades of HPMC are available. In one embodiment HPMC type 2208, preferably meeting specifications set forth in a standard pharmacopeia such as USP 24, is used. HPMC type 2208 contains 19-24% by weight methoxy and 4-12% by weight hydroxypropoxy substituents. Especially suitable HPMCs have nominal viscosity ranging from about 100 to about 10,000 mPa s; illustratively a suitable HPMC type 2208 is one having a nominal viscosity of about 4,000, with a measured viscosity of about 3,000 to about 5,600 mPa s. Such an HPMC is available, for example, as Methocel® K4MP from Dow Chemical Co., and substantially equivalent products are available from other manufacturers.

The amount of hydrophilic polymer in the composition depends on the particular polymer selected, on the active pharmaceutical agent and on the desired sustained release profile. Typically, however, the hydrophilic polymer is included in an amount of about 20% to about 70%, preferably about 30% to about 60% and more preferably about 35% to about 50%, by weight of the composition. In the illustrative case of HPMC type 2208, a suitable amount will generally be found in the range from about 30% to about 60%, preferably about 35% to about 50%, for example about 40%, by weight of the composition.

It is believed, without being bound by theory, that the hydrophilic polymer functions to provide extended or sustained release of the active pharmaceutical agent, for example by gradual dissolution or erosion of the polymer in the gastrointestinal tract.

Starches useful herein include starches from any suitable botanical source, for example corn, wheat, rice, tapioca, potato, etc., so long as they meet the requirement herein that their tensile strength is at least about 0.15 kN cm⁻² at a solid fraction representative ofthe tablet. Preferred starches have a relatively high amylose/amylopectin ratio, containing for example at least about 20%, more preferably at least about 25%, amylose. Especially preferred is pregelatinized starch, which is a type of modified starch that has been processed to render the starch more flowable and directly compressible. Partially or wholly pregelatinized starches can be used.

It is believed, without being bound by theory, that the primary function of the starch in a composition of the invention is as a binding agent. A starch meeting the tensile strength criterion defined herein can be referred to as a “super binder”.

The amount of starch in the composition is typically higher than is conventionally present as a binder in tablet formulations. Suitable amounts will generally be found in the range of about 25% to about 75% by weight. Preferably the amount of starch is about 40% to about 70%, more preferably about 45% to about 65%, for example about 50%, by weight of the composition.

Tensile strength of a starch sample can be measured by any suitable test. Illustrative test procedures are described by Hiestand & Smith (1984), Powder Technology 38, 145-159, and by Hiestand & Smith (1991), International Journal of Pharmaceutics 67, 231-246, these articles being incorporated herein by reference.

An example of a tensile strength test that can be used (herein referred to as a “triaxial tensile strength test”) requires preparation of a series of compacts of the starch sample, followed by determination oftensile strength of the compacts using a computerized multifunction tablet tester (MTT). The compacts are prepared with various degrees of compression force to provide compacts having a range of solid fraction. As a sustained release tablet formulation typically has a solid fraction of about 0.8, it is useful to prepare compacts approximating such a solid fraction.

Absolute density of the starch sample can be determined using a helium-air pycnometer.

A computer-controlled triaxial tablet press is used to prepare the compacts. Voltage output from the punch and die load cells of the tablet press are first zeroed. The punch and die are lubricated with magnesium stearate powder and the die assembly is placed in the press. Compression and decompression parameters are selected on the computer. The desired amount of starch to be compacted is weighed and poured into the die cavity. The resulting powder bed is leveled with a spatula. The punch is inserted into the die and the computer-controlled compression/decompression cycle is started.

Just prior to the end of the compression phase, thickness of the compact as measured by LVDT is recorded. At the end of the compression phase, the final compression force as measured by voltage of the punch load cell is recorded.

At the end of the decompression phase, the punch and die rams are retracted. The compact is removed from the die and inspected for defects, such as cracking or sticking. Cracking can be reduced by increasing decompression time. If the compact is free of defects, its length, width, thickness and weight are measured to enable calculation of apparent density. Solid fraction is calculated by dividing absolute density by apparent density.

In preparation of the MTT for tensile strength determination, a suitable software program is run. The platen is screwed to the load cell of the MTT and the tensile strength assembly is slid into the MTT opposite the platen. The load cell signal is monitored via the computer and the zero offset on the signal conditioner is adjusted to provide a positive baseline voltage as close as possible to zero. A forward velocity is selected that will generate a time constant of approximately 15 seconds (usually the velocity selected will be about 0.8 to about 1.2 mm s⁻¹).

The compact to be tested is placed in the holder of the tensile strength assembly. The motor is initiated via the computer, driving the platen toward the compact until the surface of the compact is detected, and stopping the platen a few millimeters from the compact. The oscilloscope is triggered, to record the force applied to the compact, and the motor is restarted. The platen is driven into the compact until a crack is detected, either by sight or by sound, and the motor is immediately reversed.

Peak force is recorded from the oscilloscope trace. Tensile strength is calculated from the peak force using appropriate computer software.

From several runs using compacts at a range of solid fractions around 0.8, data are plotted and tensile strength at a solid fraction of 0.8 is estimated. If the tensile strength at a solid fraction of 0.8 is about 0.15 kN cm⁻² or greater, the starch sample is deemed to be suitable for use in preparing a composition according to the invention.

It has now surprisingly been discovered that a much simpler test, one that is more amenable to implementation in a manufacturing setting, can be used to estimate tensile strength of a starch sample, in particular to determine whether the starch sample has a tensile strength of at least about 0.15 kN cm⁻² at a solid fraction representative of a desired sustained-release tablet.

According to this test, compacts of the starch sample are prepared on a standard automated tablet press under a range of compression forces. For example, a Carver press (e.g., Model 3888.1DT0000) fitted with flat-faced tooling of suitable diameter (e.g., 10/32 inch or about 0.7 cm for a 300 mg compact), operated at compression forces of about 4 to about 16 kN (about 900 to about 3600 lbf for a dwell time of at least about 4 seconds has been found to give satisfactory results. Illustratively, such compacts can be prepared at 1000, 1500, 2000 and 3000 lbf (4.45, 6.67, 8.90 and 13.34 kN). Preferably a dwell time of at least about 10 seconds, more preferably at least about 30 seconds, still more preferably at least about 60 seconds, is used. Illustratively, a dwell time of 90 seconds has been found to give satisfactory results. Weight, diameter and thickness of each compact are measured accurately (alternatively, diameter can be assumed to equal that of the tooling) to enable calculation of apparent density and hence solid fraction, absolute density having been measured as described above, for example by helium-air pycnometry.

Hardness of each compact thus prepared is then determined by any suitable tablet hardness test, for example using a Key HT 500 hardness tester. Hardness is a measure of the force required to cause crushing of the compact, and is typically expressed in units such as kiloponds (kp) or Strong-Cobb units (SCU). A hardness of about 10.2 kp or about 14.4 SCU corresponds to a force of 0.1 kN.

For present purposes it is considered that crushing strength of the compact is equivalent to tensile strength. Thus tensile strength (σ_{T}, in kN cm⁻²) can be calculated from the equation
σ_{T}=2F/πDH
where F is the force required to cause crushing (in kN), D is diameter of the compact (in cm) and H is thickness of the compact (in cm). For example, a compact of diameter 0.7 cm and thickness 0.4 cm having a hardness of 20 SCU (equivalent to a force of 0.139 kN) has a calculated tensile strength of 0.316 kN cm⁻².

The relationship between tensile strength and solid fraction is next established for the starch sample. This can be done by plotting data for tensile strength and solid fraction on a graph (solid fraction tends to increase with increasing compression force during preparation of the compact) or by performing a regression analysis. From that relationship, tensile strength at a standardized value of solid fraction can be estimated. The standardized value selected is one that is representative of the solid fraction of a desired sustained-release tablet, e.g., 0.8.

Where the material of the compact is pregelatinized starch, it has been found that tensile strength as determined in a simple test as described immediately above is surprisingly close to a “true” tensile strength measurement as determined by the triaxial tensile strength test method previously described, which in turn is essentially similar to methods known in the art such as that disclosed by Hiestand & Smith (1984), op. cit.

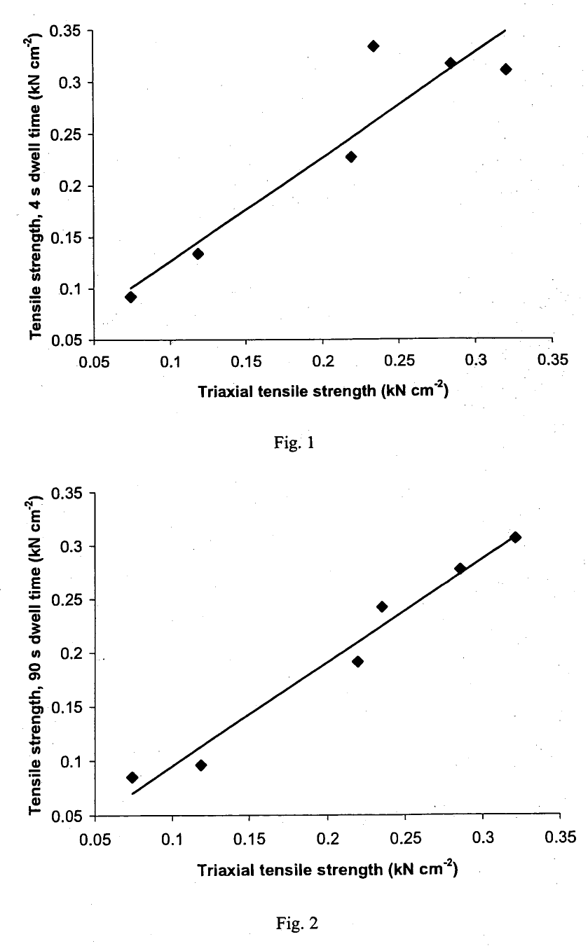

It has also been found that a longer dwell time (e.g., 90 seconds) in the test method of the present invention gives a better correlation with triaxial tensile strength than a very short dwell time (e.g., 4 seconds). See Example 1 below and FIGS. 1 and 2.

An especially preferred starch has a tensile strength of at least about 0.175 kN, even more preferably at least about 0.2 kN, at a solid fraction representative of a desired sustained-release tablet.

Even among commercially available pregelatinized starches, the preferred type of starch for use in a composition of the invention, considerable variation exists in tensile strength. Pregelatinized starches not meeting the tensile strength criterion established herein are not readily identified without testing, for example by a method as disclosed above. Such pregelatinized starches are generally unsuitable for commercial-scale manufacture of a sustained-release matrix tablet formulation as defined herein, because of a problem as set forth immediately below.

An uncoated tablet, or a tablet core prior to coating, comprising starch and a hydrophilic polymer acting as a matrix for a water-soluble drug or prodrug requires to have a certain minimum hardness in order to be able to resist breakage and/or attrition due to mechanical stresses imposed during a high-speed tableting operation (including all steps up to and including filling of the tablets into containers). The minimum acceptable hardness will depend on a number of factors, including the severity of the mechanical stresses, but is typically at least about 20 SCU, preferably at least about 22 SCU, more preferably at least about 24 SCU (about 17 kp).

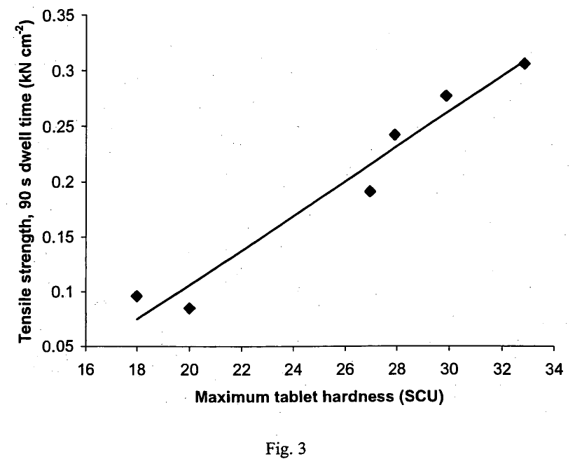

Hardness can be increased by increasing the compression force applied by the tablet press, but only up to a certain level. At least in the case of tablets as described herein, above a certain compression force, further increases in compression force give little or no firther increase in tablet hardness. There is, in other words, a maximum hardness achievable by compression of a particular starch/hydrophilic polymer/active agent composition. A starch providing a maximum hardness inadequate to withstand the mechanical stresses of a high-speed tableting and/or coating operation is unsuitable for the present purpose. As shown in FIG. 3, certain pregelatinized starches have been found to provide a maximum hardness of 20 SCU or less; these are now identified as starches having low tensile strength (0.1 kN cm⁻² or less according to the test method of the invention utilizing a dwell time of 90 seconds).

Even if a maximum hardness of at least about 20 SCU is achievable, with a starch of low tensile strength it may be achievable only by use of extremely high compression forces. A requirement for such forces reduces speed and efficiency and increases cost of a tableting operation and is undesirable for these reasons.

Where tablets are to be subjected to an additional process step after compression, in particular a coating step, exposure to mechanical stresses is further increased. According to one embodiment, therefore, the sustained-release tablet of the invention further comprises a coating, for example a nonfunctional coating. A nonfunctional coating can comprise a polymer component, for example HPMC, optionally with other ingredients, for example one or more plasticizers, colorants, etc. The term “nonfunctional” in the present context means having substantially no effect on release properties of the tablet, and should not be read to imply that the coating serves no useful purpose. For example, such a coating can impart a distinctive appearance to the tablet, provide protection against attrition during packaging and transportation, improve ease of swallowing, and/or have other benefits.

Uncoated tablets and cores of coated tablets of the invention can optionally contain one or more pharmaceutically acceptable excipients in addition to the starch and hydrophilic polymer components described above. Such excipients include without limitation glidants and lubricants. Other conventional excipients known in the art can also be included.

A glidant can be used to improve powder flow properties prior to and during tableting and to reduce caking. Suitable glidants include colloidal silicon dioxide, magnesium trisilicate, powdered cellulose, starch, talc, tribasic calcium phosphate and the like. In one embodiment, colloidal silicon dioxide is included as a glidant in an amount up to about 2%, preferably about 0.2% to about 0.6%, by weight of the tablet.

A lubricant can be used to enhance release of a tablet from apparatus on which it is formed, for example by preventing adherence to the face of an upper punch (“picking”) or lower punch (“sticking”). Suitable lubricants include magnesium stearate, calcium stearate, canola oil, glyceryl palmitostearate, hydrogenated vegetable oil, magnesium oxide, mineral oil, poloxamer, polyethylene glycol, polyvinyl alcohol sodium benzoate, sodium lauryl sulfate, sodium stearyl fumarate, stearic acid, talc, hydrogenated vegetable oil, zinc stearate and the like. In one embodiment, magnesium stearate is included as a lubricant in an amount of about 0.1% to about 1.5%, preferably about 0.3% to about 1%, by weight ofthe tablet.

Tablets can be of any suitable size and shape, for example round, oval, polygonal or pillow-shaped, and optionally bear nonfunctional surface markings. They are preferably designed to be swallowed whole and are therefore typically not provided with a breaking score. Tablets of the invention can be packaged in a container, accompanied by a package insert providing pertinent information such as, for example, dosage and administration information, contraindications, precautions, drug interactions and adverse reactions.

There is also provided a method of treatment of a subject having a condition or disorder for which a dopamine agonist is indicated, the method comprising orally administering to the subject a sustained-release pharmaceutical composition in a form of a tablet comprising as active pharmaceutical agent a compound of formula (I) or a salt thereof dispersed in a matrix comprising a hydrophilic polymer and a starch having a tensile strength of at least about 0.15 kN cm⁻² at a solid fraction representative of the tablet.

Preferably the composition is administered no more than twice daily.

Preferably the active pharmaceutical agent is a salt of sumanirole (II) or the compound of formula (III), most preferably the maleate. These agents are especially useful in treatment of Parkinson's disease, but can also be used for treatment of sexual dysfunction.

Illustratively in the case of sumanirole, suitable dosage amounts to be administered no more than twice daily include 0.5, 1, 2, 4, 8, 12 and 24 mg sumanirole in the form of sumanirole maleate.

### EXAMPLES

### Example 1

Tensile strength of six commercially obtained lots of pregelatinized starch was determined using the triaxial tensile strength test procedure described hereinabove. Data for tensile strength at a solid fraction of 0.8 are presented in Table 1.

TABLE 1
Tensile strength of pregelatinized starch lots
at a solid fraction of 0.8
(triaxial test procedure)

| Lot | Tensile strength (kN cm⁻²) |
|---|---|
| 1 | 0.323 |
| 2 | 0.220 |
| 3 | 0.074 |
| 4 | 0.119 |
| 5 | 0.287 |
| 6 | 0.236 |

A great variation in tensile strength of pregelatinized starches was observed, ranging from 0.074 to 0.323 kN cm⁻². Lots 3 and 4, exhibiting the lowest values of tensile strength, were from one manufacturer. Lots 1, 5 and 6, exhibiting the highest values of tensile strength, were from a second manufacturer. Lot 2, exhibiting an intermediate value of tensile strength, was from a third manufacturer.

### Example 2

Tensile strength of the sam six lots of pregelatinized starch was determined by the following simplified procedure.

Compacts of each starch lot were prepared on a Carver press, Model 3888.1DT0000 fitted with 10/32 inch (0.7 cm) flat-faced tooling, at compression forces of 1000, 1500, 2000 and 3000 lbf (4.45, 6.67, 8.90 and 13.34 kN), for a dwell time of 4 seconds or 90 seconds. Compacts of an additional three lots of pregelatinized starch (Lots 7, 8 and 9), from the same manufacturer as Lots 3 and 4, were prepared using a dwell time of 90 seconds only. Weight and thickness of each compact was measured (diameter being equal to that of the tooling) to enable calculation of apparent density. Absolute density of each starch lot was measured by helium-air pycnometry. Solid fraction was calculated as the ratio of apparent to absolute density.

Hardness (force required to cause crushing) of each compact was determined using a Key HT 500 hardness tester. Tensile strength was calculated from this force and dimensions of the compact, using the equation
σ_{T}=2F/πDH
as described hereinabove.

A regression analysis was performed to determine the relationship of tensile strength to solid fraction for each starch lot, and tensile strength at a standardized solid fraction of 0.8 was calculated. Data are presented in Table 2.

TABLE 2
Tensile strength of pregelatinized starch lots
at a solid fraction of 0.8
(simplified test procedure of the invention)

Tensile strength (kN cm⁻²)

| Lot | 4 s dwell time | 90 s dwell time |
|---|---|---|
| 1 | 0.310 | 0.306 |
| 2 | 0.227 | 0.191 |
| 3 | 0.092 | 0.085 |
| 4 | 0.134 | 0.096 |
| 5 | 0.316 | 0.277 |
| 6 | 0.333 | 0.242 |
| 7 | n.d. | 0.087 |
| 8 | n.d. | 0.088 |
| 9 | n.d. | 0.172 |

Correlation of tensile strength as measured in the simplified test using a 4 second dwell time (this Example) with tensile strength as measured by the triaxial test procedure of Example 1 is shown graphically in FIG. 1.

Correlation of tensile strength as measured in the simplified test using a 90 second dwell time (this Example) with tensile strength as measured by the triaxial test procedure of Example 1 is shown graphically in FIG. 2.

Both dwell times exhibited a strong correlation, but the correlation was especially close where the simplified test used a 90 second dwell time. It is concluded that the simplified test as herein described can be used to estimate tensile strength of a starch lot for the purpose of predicting whether that starch lot will be suitable for preparing a sustained-release tablet formulation of the present invention.

### Example 3

Sumanirole maleate sustained-release tablets were prepared having the compositions shown in Table 3. Tablet strength in mg is expressed as sumanirole base.

TABLE 3
Composition of sumanirole maleate tablets of Example 3

| | Tablet strength (mg) | | | | | | | |
|---|---|---|---|---|---|---|---|---|
| | 0.5 | 1 | 2 | 4 | 8 | 8 | 12 | 24 |
| Ingredient | Amount (% by weight) | | | | | | | |
| sumanirole | 0.23 | 0.45 | 0.9 | 1.8 | 3.6 | 3.6 | 5.4 | 10.9 |
| maleate | | | | | | | | |
| HPMC type | 35.00 | 35.00 | 35.0 | 35.0 | 35.0 | 35.0 | 35.0 | 35.0 |
| 2208, 4000 | | | | | | | | |
| mPa s | | | | | | | | |
| pregelatinized | 63.87 | 63.65 | 63.2 | 62.3 | 60.5 | 60.0 | 58.2 | 52.5 |
| starch | | | | | | | | |
| colloidal | 0.40 | 0.40 | 0.4 | 0.4 | 0.4 | 0.4 | 0.4 | 0.4 |
| silicon | | | | | | | | |
| dioxide | | | | | | | | |
| magnesium | 0.50 | 0.50 | 0.5 | 0.5 | 0.5 | 1.0 | 1.0 | 1.0 |
| stearate | | | | | | | | |

All ingredients except the lubricant (magnesium stearate) were screened to remove lumps and were blended thoroughly in a low-shear mixer operating at 24 rpm for 10-30 minutes. The lubricant was then screened into the mixer and the materials were blended for a further 2-5 minutes. The resulting lubricated mixture was compressed into 350 mg pillow-shaped tablets using a Kilian S100 tableting machine.

### Example 4

Tablets similar to those of Example 3 were prepared using pregelatinized starches of lots 1-6 as tested in Examples 1 and 2. Maximum hardness of the tablets obtainable with each pregelatinized starch lot was determined.

Maximum hardness was correlated with tensile strength of the pregelatinized starch lot used, as measured in the simplified test of Example 2 using a 90 second dwell time. Results are shown in FIG. 3. The correlation was substantially linear.

In subsequent tests, tablets of different hardness were used as cores for coating and were tested for resistance to erosion during a high-speed coating operation. Tablet cores having a hardness of at least about 24 SCU (about 17 kp) were found to have acceptable resistance to erosion. As shown in FIG. 3, this degree of hardness is achievable using pregelatinized starch having a tensile strength of at least about 0.175 kN cm⁻². Pregelatinized starches of Lots 3 and 4 were unsuitable, having tensile strength less than about 0.15 kN cm⁻² and providing tablets having a maximum hardness no greater than about 20 SCU (about 14 kp).

1. A pharmaceutical composition in a form of an orally deliverable tablet comprising as active pharmaceutical agent a compound of formula 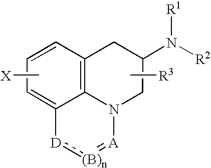
or a pharmaceutically acceptable salt thereof, wherein
R¹, R² and R³ are the same or different and are H, C₁₋₆ alkyl (optionally phenyl substituted), C₃₋₅ alkenyl or alkynyl or C₃₋₁₀ cycloalkyl, or where R³ is as above and R¹ and R² are cyclized with the attached N atom to form pyrrolidinyl, piperidinyl, morpholinyl, 4-methylpiperazinyl or imidazolyl groups;
X is H, F, Cl, Br, I, OH, C₁₋₆ alkyl or alkoxy, CN, carboxamide, carboxyl or (C₁₋₆ alkyl)carbonyl;
A is CH, CH₂, CHF, CHCl, CHBr, CHI, CHCH₃, C═O, C═S, CSCH₃, C═NH, CNH₂, CNHCH₃, CNHCOOCH₃, CNHCN, SO₂ or N;
B is CH, CH₂, CHF, CHCl, CHBr, CHI, C═O, N, NH or NCH₃, and n is 0 or 1; and
D is CH, CH₂, CHF, CHCl, CHBr, CHI, C═O, O, N, NH or NCH₃;
said compound or salt thereof being dispersed in a matrix comprising a hydrophilic polymer and a starch having a tensile strength of at least about 0.15 kN cm⁻² at a solid fraction representative ofthe tablet.

2. The composition of claim 1 wherein the starch has a tensile strength of at least about 0.175 kN cm⁻².

3. The composition of claim 1 wherein the starch has a tensile strength of at least about 0.2 kN cm⁻².

4. The composition of claim 1 wherein the starch is a pregelatinized starch.

5. The composition of claim 1 wherein the starch is present in an amount of about 25% to about 75% by weight.

6. The composition of claim 1 wherein the starch is present in an amount of about 40% to about 70% by weight.

7. The composition of claim 1 wherein the starch is present in an amount of about 45% to about 65% by weight.

8. The composition of claim 1 wherein the hydrophilic polymer is selected from the group consisting of methylcellulose, HPMC, carmellose sodium and carbbmer.

9. The composition of claim 1 wherein the hydrophilic polymer is HPMC.

10. The composition of claim 1 wherein the hydrophilic polymer is present in an amount of about 20% to about 70% by weight.

11. The composition of claim 1 wherein the hydrophilic polymer is present in an amount of about 30% to about 60% by weight.

12. The composition of claim 1 wherein the hydrophilic polymer is present in an amount of about 35% to about 50% by weight.

13. The composition of claim 1 wherein the active pharmaceutical agent has solubility not less than about 10 mg/ml.

14. The composition of claim 1 wherein the active pharmaceutical agent has solubility not less than about 50 mg/ml.

15. The composition of claim 1 wherein the active pharmaceutical agent has solubility not less than about 100 mg/ml.

16. The composition of claim 1 wherein the active pharmaceutical agent is a salt of sumanirole.

17. The composition of claim 1 wherein the active pharmaceutical agent is sumanirole maleate.

18. The composition of claim 17 that comprises about 0.5 to about 25 mg sumanirole per tablet.

19. The composition of claim 17 that comprises about 0.5, 1, 2, 4, 8, 12 or 24 mg sumanirole per tablet.

20. The composition of claim 1 wherein the active pharmaceutical agent is a salt of (R)-5,6-dihydro-5-(methylamino)-4H-imidazo[4,5-ij]-quinoline-2(1H)-thione.

21. The composition of claim 1 wherein the active pharmaceutical agent is the maleate salt of (R)-5,6-dihydro-5-(methylamino)-4H-imidazo[4,5-ij]-quinoline-2(1H)-thione.

22. A pharmaceutical composition in a form of an orally deliverable tablet comprising sumanirole maleate in an amount of about 0.5, 1, 2, 4, 8, 12 or 24 mg, dispersed in a matrix comprising (a) HPMC type 2208 in an amount of about 35% to about 50% by weight of the tablet and (b) a pregelatinized starch having a tensile strength of at least about 0.15 kN cm⁻² at a solid fraction of 0.8, in an amount of about 45% to about 65% by weight of the tablet.

23. A method of treatment of a subject having a condition or disorder for which a dopamine agonist is indicated, the method comprising orally administering to the subject the pharmaceutical composition of claim 1.

24. The method of claim 23 wherein the composition is administered no more than once daily.

25. The method of claim 23 wherein the condition or disorder is Parkinson's disease.

26. The method of claim 23 wherein the condition or disorder is sexual dysfunction.

27. The method of claim 23 wherein the composition is in a form of an orally deliverable tablet comprising sumanirole maleate in an amount of about 0.5, 1, 2, 4, 8, 12 or 24 mg, dispersed in a matrix comprising (a) HPMC type 2208 in an amount of about 35% to about 50% by weight of the tablet and (b) a pregelatinized starch having a tensile strength of at least about 0.15 kN cm⁻² at a solid fraction of 0.8, in an amount of about 45% to about 65% by weight of the tablet.

28. A process for preparing a sustained-release pharmaceutical composition in a form of an orally deliverable tablet, the process comprising selecting by a suitable test a starch having a tensile strength of at least about 0.15 kN cm⁻² at a solid fraction representative of the tablet; admixing with the selected starch a hydrophilic polymer and an active pharmaceutical agent that is a compound of formula 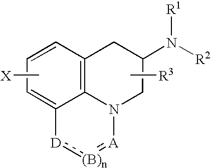
or a pharmaceutically acceptable salt thereof, wherein
R¹, R² and R³ are the same or different and are H, C₁₋₆ alkyl (optionally phenyl substituted), C₃₋₅ alkenyl or alkynyl or C₃₋₁₀ cycloalkyl, or where R³ is as above and R¹ and R² are cyclized with the attached N atom to form pyrrolidinyl, piperidinyl, morpholinyl, 4-methylpiperazinyl or imidazolyl groups;
X is H, F, Cl, Br, I, OH, C₁₋₆ alkyl or alkoxy, CN, carboxamide, carboxyl or (C₁₋₆ alkyl)carbonyl;
A is CH, CH₂, CHF, CHCl, CHBr, CHI, CHCH₃, C═O, C═S, CSCH₃, C═NH, CNH₂, CNHCH₃, CNHCOOCH₃, CNHCN, SO₂ or N;
B is CH, CH₂, CHF, CHCl, CHBr, CHI, C═O, N, NH or NCH₃, and n is 0 or 1; and
D is CH, CH₂, CHF, CHCl, CHBr, CHI, C═O, O, N, NH or NCH₃;
to provide a rnixture wherein the agent is dispersed in a matrix comprising the polymer and the starch; and compressing the mixture to form said tablet.
